(19) United States
(12) Patent Application Publication
(10) Pub. No.: US 2025/0387033 A1
(43) Pub. Date: Dec. 25, 2025

(54) BLOOD PRESSURE DETERMINATION USING PRESSURE PERTURBATION

(71) Applicant: Meta Platforms Technologies, LLC, Menlo Park, CA (US)

(72) Inventor: Paurakh RAJBHANDARY, San Jose, CA (US)

(21) Appl. No.: 18/332,611

(22) Filed: Jun. 9, 2023

Related U.S. Application Data
(60) Provisional application No. 63/351,985, filed on Jun. 14, 2022.
(60) Provisional application No. 63/383,227, filed on Nov. 10, 2022.

(51) Int. Cl. A61B 5/0225; A61B 5/00; A61B 5/021

(52) U.S. Cl. CPC A61B 5/02255; A61B 5/02108; A61B 5/7278; A61B 2562/0247

(57) ABSTRACT

A method for determining blood pressure is provided. The method may involve obtaining measured plethysmograph data and pressure data that corresponds to a time period comprising a first portion during which an external pressure perturbation was not applied and a second portion during which the external pressure perturbation was applied. The method may involve generating predicted plethysmograph data using: (i) initial blood pressure values; (ii) at least a subset of the measured plethysmograph data associated with the first portion during which the external pressure perturbation was not applied; and (iii) the pressure data. The method may involve modifying the initial blood pressure values based on a difference between the predicted plethysmograph data and the measured plethysmograph data to generate final blood pressure values.

### CROSS-REFERENCE TO RELATED APPLICATIONS

This application claims priority under 35 U.S.C. § 119 to U.S. Provisional Application 63/351,985 filed on Jun. 14, 2022 and U.S. Provisional Application No. 63/383,227, filed on Nov. 10, 2022, the contents of each of which are hereby incorporated by reference in their entirety for all purposes.

### BACKGROUND

Accurate and non-invasive blood pressure measurements may be important, for example, to monitor cardiovascular heath, quickly identify blood pressure abnormalities that may portend a serious health issue, etc. Because blood pressure can vary substantially over the course of the day, quick and accurate measurements of blood pressure may be useful to allow people to monitor and track their health. However, accurate measurements of blood pressure, that are measured quickly and non-invasively, are difficult to obtain.

### SUMMARY

Disclosed herein are methods, systems, and media for blood pressure determination using pressure perturbation.

In some embodiments, a method of determining blood pressure comprises: obtaining measured plethysmograph data and pressure data that corresponds to a time period comprising a first portion during which an external pressure perturbation was not applied and a second portion during which the external pressure perturbation was applied; generating predicted plethysmograph data using: (i) initial blood pressure values; (ii) at least a subset of the measured plethysmograph data associated with the first portion during which the external pressure perturbation was not applied; and (iii) the pressure data; and modifying the initial blood pressure values based on a difference between the predicted plethysmograph data and the measured plethysmograph data to generate final blood pressure values.

In some examples, generating the predicted plethysmograph comprises: generating a synthetic arterial pressure waveform based on at least a subset of the measured plethysmograph data associated with the first portion during which the external pressure perturbation was not applied and using initial blood pressure values, wherein the synthetic arterial pressure waveform represents an internal blood vessel pressure without any external pressure perturbation; and modifying the synthetic arterial pressure waveform to generate a transmural pressure waveform using at least the pressure data, wherein the predicted plethysmograph data is generated using the transmural pressure waveform. In some examples, generating the synthetic arterial pressure waveform comprises: generating synthetic plethysmograph data based on the subset of the plethysmograph data associated with the first portion during which the external pressure perturbation was not applied; and scaling the synthetic plethysmograph data using the initial blood pressure values. In some examples, generating the predicted plethysmograph data comprises applying the transmural pressure waveform to a blood vessel elasticity model that generates, for a given transmural pressure, a corresponding blood volume of the blood vessel.

In some examples, the predicted plethysmograph data indicates changes to a shape of a cycle within the plethysmograph data due to the external pressure perturbation.

In some examples, the first portion during which the external pressure perturbation is not applied is identified using data from at least one motion sensor.

In some examples, modifying the initial blood pressure values based on the difference between the predicted plethysmograph data and the measured plethysmograph data comprises using an iterative optimization algorithm to obtain the final blood pressure values.

In some examples, the external pressure perturbation comprises at least one of: an externally applied compression force; or a change in hydrostatic pressure.

In some examples, the measured plethysmograph data is obtained using at least one light source and light detector disposed in or on a wearable device, and wherein the pressure data is obtained from at least one force sensor disposed in or on the wearable device.

In some examples, the method further involves prior to obtaining the measured plethysmograph data and the pressure data, presenting an instruction to a user to apply the external pressure perturbation.

In some examples, the time period is less than about 5 seconds.

In some embodiments, a method of determining blood pressure comprises: obtaining measured plethysmograph data of a user and hydrostatic pressure data that corresponds to a time period comprising a first portion during which a change in hydrostatic pressure was not applied and a second portion during which the change in the hydrostatic pressure was applied; and determining a blood pressure of the user based on the measured plethysmograph data and the hydrostatic pressure data.

In some examples, the change in the hydrostatic pressure is due to an elevational change in a hydrostatic pressure sensor. In some examples, the hydrostatic pressure sensor is disposed in a wrist-worn device, and wherein the elevational change is due to the wrist-worn device being lifted. In some examples, the method further involves presenting instructions to the user to lift the wrist-worn device to cause the change in the hydrostatic pressure. In some examples, the method further involves detecting the change in the hydrostatic pressure without instructing the user to cause the change in the hydrostatic pressure, wherein the plethysmograph data is measured responsive to detecting the change in the hydrostatic pressure.

In some embodiments, a wearable device comprises: one or more plethysmograph sensors configured for obtaining plethysmograph data from a wearer of the wearable device; one or more pressure sensors configured for obtaining hydrostatic pressure data; and a controller. The controller may be configured to: obtain measured plethysmograph data of the wearer using the one or more plethysmograph sensors and hydrostatic pressure data using the one or more pressure sensors, wherein the measured plethysmograph data and the measured hydrostatic pressure data correspond to a time period comprising a first portion during which a change in hydrostatic pressure was not applied and a second portion during which the change in the hydrostatic pressure was applied; and determine a blood pressure of the wearer based on the measured plethysmograph data and the hydrostatic pressure data.

In some examples, the wearable device is a wrist-worn device, and wherein the elevational change is due to the wrist-worn device being lifted. In some examples, the controller is further configured to cause instructions to lift the wrist-worn device on a display of the wearable device.

In some examples, the controller is configured to determine the blood pressure by: generating a predicted plethysmograph waveform based at least on a subset of the measured plethysmograph data from the first portion of the time period during which the change in hydrostatic pressure was not applied; and determining the blood pressure based on a comparison of the predicted plethysmograph waveform to the measured plethysmograph data over the time period.

### BRIEF DESCRIPTION OF THE DRAWINGS

Illustrative embodiments are described in detail below with reference to the following figures.

FIG. 1 illustrates graphs that depict usage of the oscillometric technique for determining blood pressure according to certain embodiments.

FIG. 2 is a schematic diagram of a portion of a wearable device that may be used for blood pressure determination according to certain embodiments.

FIG. 3 is a flowchart of an example process for determining blood pressure using pressure perturbation according to certain embodiments.

FIG. 4 illustrates example graphs of measured plethysmography data and measured pressure data according to certain embodiments.

FIG. 5 illustrates an example graph that depicts measured plethysmography data and a synthetic plethysmography waveform according to certain embodiments.

FIG. 6 illustrates an example arterial pressure waveform according to certain embodiments.

FIG. 7 illustrates example graphs that depict determination of a transmural pressure waveform according to certain embodiments.

FIG. 8A and 8B illustrates example graphs associated with a blood vessel elasticity model that may be used in certain embodiments.

FIG. 9 illustrates a measured plethysmography waveform and a corresponding predicted plethysmography waveform according to certain embodiments.

FIG. 10 is a simplified block diagram of an example of a computing system that may be implemented as part of a user device according to certain embodiments.

FIG. 11 is a simplified block diagram of an example of a computing system that may be implemented as part of a server according to certain embodiments.

The figures depict embodiments of the present disclosure for purposes of illustration only. One skilled in the art will readily recognize from the following description that alternative embodiments of the structures and methods illustrated may be employed without departing from the principles, or benefits touted, of this disclosure.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Accurate and non-invasive blood pressure measurements may be important, for example, to monitor cardiovascular heath, quickly identify blood pressure abnormalities that may portend a serious health issue, etc. Because blood pressure can vary substantially over the course of the day, quick and accurate measurements of blood pressure may be useful to allow people to monitor and track their health. Accordingly, being able to measure blood pressure via a wearable device, such as a smart watch, a ring, a patch, a head mounted device, smart glasses, or the like, is desirable. However, most techniques that have been attempted to measure blood pressure in wearable devices have struggled to measure blood pressure accurately.

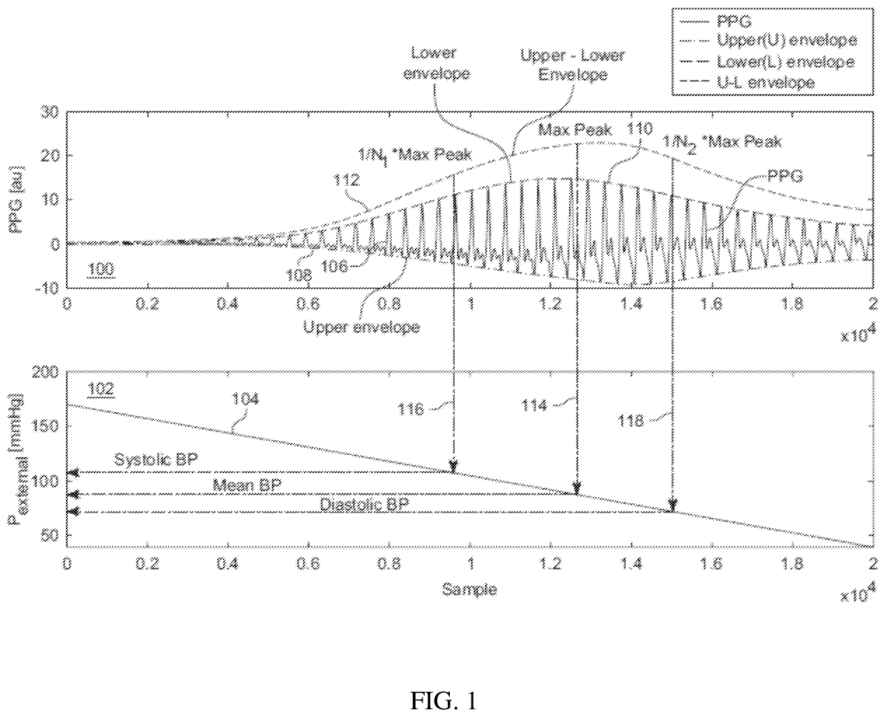

The oscillometric technique is a conventional technique for non-invasively determining blood pressure. In the oscillometric technique, an external pressure is applied to occlude passage of blood through an artery. For example, the external pressure may be applied by a cuff on the upper arm, as is conventionally used in, e.g., a doctor's office. As another example, in some cases, the external pressure may be applied by a user applying a force by, e.g., pressing their finger or other appendage to block blood flow. After the external pressure has been increased to the point where blood flow is completely occluded, the external pressure is slowly released over a time period of, e.g., 10-30 seconds. When blood flow is completely impeded, or when there is no external pressure, arterial pressure on the vessel wall will be essentially normal. However, during the intermediate period of external pressure release (e.g., where there is some blood flow and external pressure application), outward pressure on the vessel wall will be increased. In some cases, the oscillometric technique may measure the outward pressure on the vessel wall (e.g., the arterial pressure) by measuring an outward pressure, e.g., on the cuff that is applying the external pressure. Arterial pressure on the vessel wall may cause changes in the absolute value of blood volume in the artery, which may be evidenced by an oscillation in the amplitude of a plethysmography signal. Accordingly, in some applications of the oscillometric technique, the arterial pressure on the vessel wall may be measured by measuring the oscillations in the amplitude of the plethysmography signal, which is shown in and described below in more detail in connection with FIG. 1.

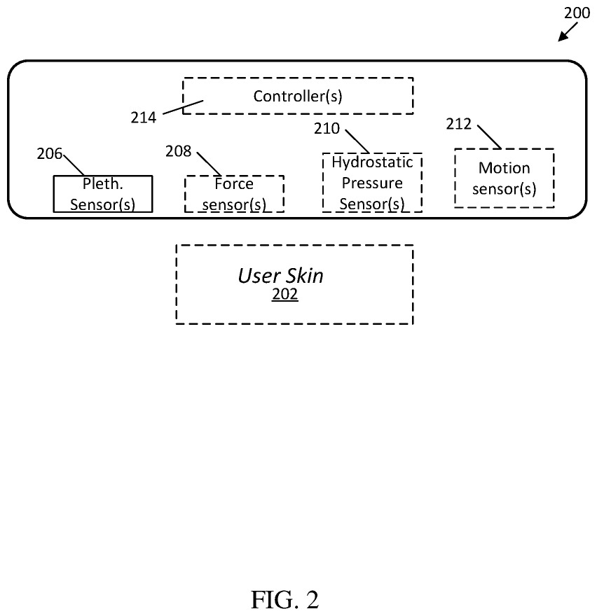

FIG. 1 illustrates use of the oscillometric technique using PPG. Graph 100 illustrates a measured PPG waveform, and graph 102 illustrates the magnitude of an applied external pressure (e.g., as applied by a cuff, applied by a person pressing an appendage such as a finger against a surface, or the like). As described above, the magnitude of the external pressure (Pexternal) is increased to a point where blood flow through the artery is completely impeded. The external pressure is then released, as shown by curve 104 in graph 102. While the external pressure is being released, photoplethysmograph (PPG) data is collected, as shown by curve 106. The trough of each cycle of the PPG waveform is shown by curve 108, and the peak of each cycle of the PPG waveform is shown by curve 110. The difference between the peak and the trough, for each cycle, is shown by curve 112, which represents the amplitude of a single cycle of the PPG waveform as a function of time. As shown in graph 100, as the external pressure is released, the amplitude of the PPG waveform (e.g., as shown in curve 112) increases to a maximum, and then decreases. The external pressure magnitude corresponding to the PPG waveform maximum (as shown by dropline 114) corresponds to the mean blood pressure. The systolic blood pressure and the diastolic blood pressure may be determined based on heuristic values with respect to the value of the maximum of the PPG amplitude. For example, the systolic blood pressure may be determined as the external pressure corresponding to a first predetermined fraction of the maximum of the PPG amplitude that occurs prior to the maximum of the PPG amplitude (denoted in FIG. 1 as 1/N₁*Max_{Peak}), as indicated by drop line 116. As another example, the diastolic blood pressure may be determined as the external pressure corresponding to a second predetermined fraction of the maximum of the PPG amplitude that occurs after the maximum of the PPG amplitude (denoted in FIG. 2 as 1/N₂*Max_{Peak}), as indicated by drop line 118.

Because the oscillometric technique requires increasing the external pressure until blood flow is completely impeded and subsequently decreasing the external pressure at a rate slow enough to capture oscillations in arterial vessel pressure and/or the corresponding PPG amplitude oscillations, measurement of blood pressure using the oscillometric technique may require on the order of 20-30 seconds. Moreover, the oscillometric technique requires application of an external force sufficient to completely block or impede blood flow in an artery. These factors make the oscillometric technique difficult to utilize in a wearable user device, such as a smart watch, a ring, a patch, a head mounted device, smart glasses, etc. For example, an untrained user (e.g., not a medical professional) may have trouble applying the correct amount of pressure, and appropriately varying the pressure, over the 20-30 seconds required to obtain the blood pressure measurement.

Disclosed herein are systems, methods, and techniques for determining blood pressure using plethysmography and external pressure perturbation. In particular, the techniques described herein may be used to determine blood pressure using plethysmography samples collected over a substantially shorter time duration (e.g., within about 1-10 seconds) relative to data collected to perform the oscillometric technique described above in connection with FIG. 1. Note that, in some cases, the techniques described herein may additionally or alternatively be performed over other time frames, such as within a range of about 10 seconds-60 seconds, or the like. The techniques described herein utilize plethysmograph data collected with and without an external pressure perturbation. The external pressure perturbation may be a force applied to a body region on which the plethysmograph data is applied sufficient to at least partially impede blood flow, and/or a change in hydrostatic pressure (e.g., induced by a change in vertical position of the body region). A predicted plethysmograph waveform may be generated based on initial blood pressure values and a portion of the plethysmograph data collected during a time period during which the external pressure perturbation was not applied, where the predicted plethysmograph waveform is intended to predict the measured plethysmograph data collected over the entire time period that includes the external pressure perturbation. The initial blood pressure values are then modified using an optimization algorithm in order to minimize a difference between the measured plethysmograph data and the predicted plethysmograph waveform. In other words, the external pressure perturbation is used to induce a change in the plethysmograph data relative to plethysmograph data without the external pressure perturbation, where the difference between the plethysmograph data with and without the external pressure perturbation can be used to determine a physiological blood pressure of the user that would induce such a difference. It should be noted that, during the time period over which the external pressure perturbation is applied, the blood pressure of the person stays substantially constant (e.g., within about +/−5%, within about +/−10%, or the like).

In some implementations, the predicted plethysmograph waveform is generated by generating an arterial pressure waveform that represents outward pressure on the blood vessel walls. The arterial pressure waveform may be determined using the initial blood pressure values and a synthetic plethysmograph waveform that represents hypothetical plethysmograph data without any external pressure perturbation. The arterial pressure waveform may then be used in conjunction with pressure data (measured concurrently with the measured plethysmograph data) to determine a transmural pressure waveform. The transmural pressure may represent a net pressure on the blood vessel walls. The predicted plethysmograph waveform may then be determined using the transmural pressure, e.g., by utilizing a blood vessel elasticity model.

The blood pressure may be determined using a wearable device that includes multiple sensors. For example, the sensors may include one or more sensors for obtaining plethysmograph data, which may include photoplethysmograph (PPG) data, impedance plethysmograph data, or the like. As another example, the sensors may include one or more sensors for obtaining force measurements, such as compression force and/or strain force on the wearable device. As yet another example, the sensors may include one or more sensors for obtaining hydrostatic pressure measurements. As still another example, the sensors may include one or more motion sensors, which may be used to identify portions of the measured plethysmograph data during which the user was not in motion and/or during which there was no external pressure perturbation. The one or more sensors may all be packaged in the wearable device, such as within a capsule of a smart watch, or the like.

FIG. 2 is a schematic diagram of a portion of an example wearable device 200 that may be used for determining blood pressure using external pressure perturbations. Wearable device 200 may be any suitable type of wearable device, such as a wrist-worn device (e.g., a smart watch, a bracelet, etc.), a ring, a head-mounted device, smart glasses, a wrap configured to be worn around an appendage (e.g., a leg, an arm, a chest strap, etc.), or the like. As illustrated, a portion of wearable device 200 may be configured proximate to the user's skin 202. In some implementations, wearable device 200 may include one or more plethysmography sensors 206. As used herein, plethysmography refers to a technique for measuring blood volume, or changes in blood volume as a function of time, in a vessel. Plethysmography sensors 206 may include sensors and/or emitters used to collect plethysmography data. For example, plethysmography sensors 206 may include one or more light emitters and one or more light detectors configured for performing photoplethysmography. Continuing with this example, the one or more light emitters may be configured to emit light toward user skin 202, and the one or more light detectors may be configured to detect reflected light (e.g., reflected from user skin 202, reflected from internal portions of the user's body, etc.). In some embodiments, plethysmography sensors 206 may include sensors configured to perform impedance plethysmography. For example, the one or more sensors may include one or more electrodes configured to measure an electrical current in the body region of the user. Continuing with this example, the electrical current may be utilized to perform impedance plethysmography, for example, by determining changes in resistance in the body region as a function of time. Plethysmography sensors 206 may include any other suitable type of plethysmography suitable for obtaining data usable to measure blood volume changes in underlying vessels within a body region of a wearer of wearable device 200.

Wearable device 200 may include one or more force sensor(s) 208. Force sensor(s) 208

may be configured to measure an externally applied force. The externally applied force may include a user pressing on an external casing of wearable device 200, such as a watch face of a wearable watch, a frame of wearable device 200, or the like. Force sensor(s) 208 may be configured to measure a compression force or a strain (e.g., bending) force. Wearable device 200 may include one or more hydrostatic pressure sensor(s) 210. Hydrostatic pressure sensor(s) 210 may be configured to measure changes in hydrostatic pressure induced by, e.g., a change in vertical position of wearable device 200.

Wearable device 200 may include one or more motion sensor(s) 212. Motion sensor(s) 212 may include one or more accelerometers, one or more magnetometers, or the like. In some implementations, motion sensor(s) 212 may be configured to collect measurements at any suitable interval (e.g., every five seconds, every ten seconds, every thirty seconds, every minute, etc.) suitable to determine whether a wearer is in motion or not.

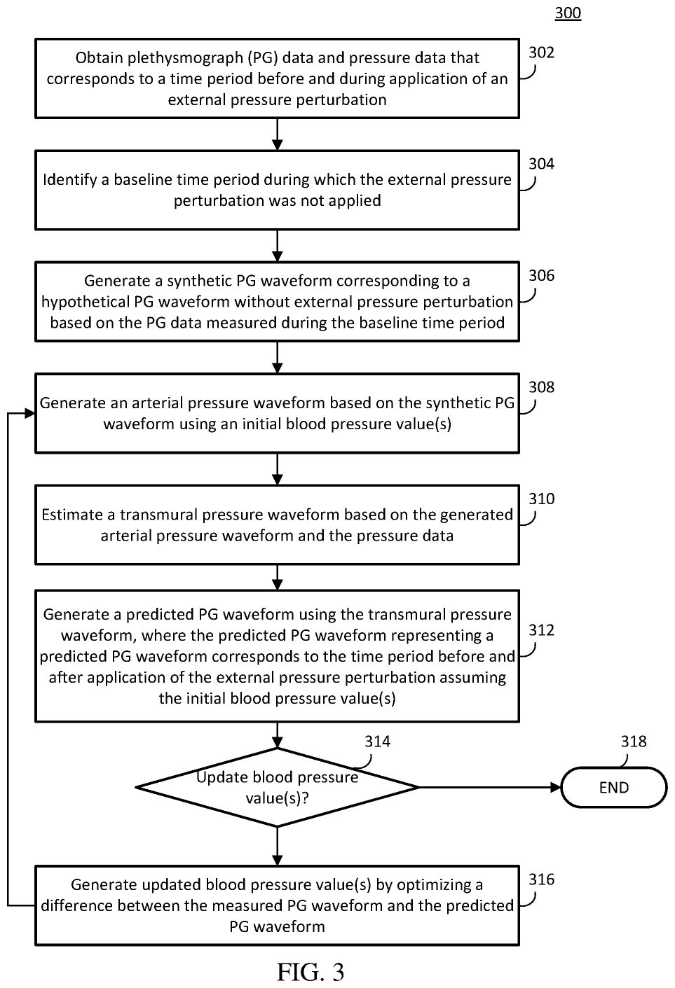

Wearable device 200 may include a controller 214. Controller 214 may be configured to utilize data from any of sensors 206-212 to determine a blood pressure of a wearer of wearable device 200. For example, in some implementations, controller 214 may be configured to implement blocks of process 300 shown in and described below in connection with FIG. 3 to determine the blood pressure.

In some embodiments, blood pressure may be determined by measuring plethysmograph data and external pressure data concurrently, where the plethysmograph data and the external pressure data are measured during a first time period where no external pressure perturbation is applied, and during a second time period where an external pressure perturbation is applied. The external pressure perturbation may be a change in force applied to a body region at which the plethysmograph data is being applied. For example, in an instance in which the plethysmograph data is obtained from a wrist region of a person (e.g., from sensors disposed in or on a wrist-worn device), the force may be a force applied to the wrist region by, e.g., pressing on an outer capsule of the wrist-worn device. In some embodiments, the external pressure perturbation may be a change in hydrostatic pressure. For example, the change in hydrostatic pressure may be induced by the body region at which the plethysmograph data is obtained being lifted in a vertical direction.

In some embodiments, the blood pressure may be determined by generating predicted plethysmograph data using initial blood pressure values (e.g., an initial systolic value and an initial diastolic value), the measured plethysmograph data collected during the time period during which no external pressure perturbation was applied, and the pressure data indicating magnitude of the external pressure applied. For example, in some embodiments, the blood pressure may be determined based on a difference between the predicted plethysmograph data and the measured plethysmograph data. As a more particular example, in some embodiments, an optimization algorithm may be used to determine blood pressure values that minimize a difference between the predicted plethysmograph data and the measured plethysmograph data. In other words, the predicted plethysmograph data may indicate a blood pressure that, when combined with the external pressure perturbation, causes plethysmograph data measured without an external pressure perturbation to have the measured plethysmograph characteristics.

In some implementations, the predicted plethysmograph data may be generated by generating a synthetic arterial pressure waveform that indicates an internal blood vessel pressure in the absence of external pressure perturbation. For example, the synthetic arterial pressure waveform may be generated by utilizing at least a subset of the measured plethysmograph data from a period of time during which no external pressure perturbation was applied and scaling the subset of the measured plethysmograph data using initial blood pressure values. A transmural pressure waveform may then be generated using the synthetic arterial pressure waveform, where the transmural pressure waveform indicates a difference between the synthetic arterial pressure waveform (e.g., which indicates outward pressure on the blood vessels) and the external pressure (e.g., which indicates pressure applied on the blood vessels externally). In some embodiments, the predicted plethysmograph data may be generated from the transmural pressure using a blood vessel compliance model.

It should be noted that the blood pressure may be determined using measured plethysmograph data that spans substantially less than the time duration required to perform the oscillometric technique. For example, the measured plethysmograph data may be collected over 1 second, 3 seconds, 5 seconds, 10 seconds, or the like, whereas the oscillometric technique may require 20-30 seconds of plethysmograph data. Moreover, the external pressure perturbation used in the techniques disclosed herein need not completely occlude blood flow in the artery, unlike in the oscillometric technique. In addition, the predicted plethysmograph data may include changes to the shape (e.g., shape characteristics other than amplitude) of a single cycle or a relatively small number of cycles (e.g., two cycles, three cycles, five cycles, or the like) in the plethysmograph data due to the external pressure perturbation. The morphology of the cycle in the plethysmograph data may implicitly indicate various cardiovascular characteristics, and moreover, is additionally affected by the blood pressure through contributions to the transmural pressure.

Accordingly, the techniques disclosed herein may allow morphology characteristics implicitly included in shape features of the plethysmograph data to be utilized in determining blood pressure.

FIG. 3 is a flowchart of an example process 300 for determining blood pressure using external pressure perturbations in accordance with some embodiments. In some implementations, blocks of process 300 may be implemented using a controller or a processor of a wearable device, as shown in and described above in connection with FIG. 2. In some embodiments, blocks of process 300 may be performed in an order other than what is shown in FIG. 3. In some implementations, two or more blocks of process 300 may be performed substantially in parallel. In some implementations, one or more blocks of process 300 may be omitted. It should be noted that although many of the examples described below in connection with FIG. 3 utilize example

PPG waveforms and data, the techniques described below in connection with FIG. 3 may be utilized in connection with other types of plethysmography, such as impedance plethysmography.

Process 300 can begin at block 302 by obtaining plethysmograph data and pressure data that corresponds to a time period before and during application of an external pressure perturbation. The plethysmograph data may include any type of plethysmograph data, or any combination of types of plethysmograph data. For example, the plethysmograph data may include PPG data measured using one or more light emitters and one or more light detectors, impedance plethysmograph data, and/or a combination. The plethysmograph data may be collected using one or more plethysmograph sensors, as shown in and described above in connection with FIG. 2. The pressure data may be collected using one or more force sensor(s) and/or one or more hydrostatic pressure sensor(s), as shown in and described above in connection with FIG. 2.

In some embodiments, the external pressure perturbation may include an applied force on a body region from which the plethysmograph data is measured. For example, in an instance in which the plethysmograph data is measured from a wrist region of the user, the applied force may include a compression force on the wrist region. As a more particular example, in an instance in which the plethysmograph data is measured using one or more sensors disposed proximate to a backside of a wrist-worn device such as a smart watch, the applied force may include the user pressing on the top side of the wrist-worn device (e.g., opposite to the surface on which the one or more sensors are disposed) to exert a compression force on the wrist region. Similar external forces may be applied for other body regions, such as near a finger, near a forehead, near the car, etc. for other types of wearable devices. In some embodiments, the external pressure perturbation may include a change in hydrostatic pressure. For example, the change in hydrostatic pressure may be induced by the body region on which the plethysmograph data is measured being vertically lifted. As a more particular example, in an instance in which the body region is a wrist of the user, a change in hydrostatic pressure may be induced by the user lifting their wrist. It should be noted that, in some implementations, the external pressure perturbation may include both an applied force and a change in hydrostatic pressure.

It should be noted that, in some instances, the external pressure perturbation may be applied or induced responsive to instructions being presented to the user to cause the external pressure perturbation. For example, the instructions may instruct a user to press the wearable device in a particular location to apply an external force. As a more particular example, in some embodiments, the instructions may instruct the user to press the wearable device in a particular manner, such as with an increasing force over a particular time period (e.g., over two seconds, over five seconds, etc.). As another example, the instructions may instruct a user to induce a change in hydrostatic pressure by, e.g., raising the region of their body wearing the wearable device (e.g., over their head, above their heart, or the like). Alternatively, in some implementations, the external pressure perturbation may occur without explicit user instruction. For example, in some embodiments, process 300 may automatically detect an external pressure perturbation and may, in response, collect the plethysmograph data and the pressure data obtained at block 302. As a more particular example, in some implementations, process 300 may detect a change in hydrostatic pressure due to a user lifting their arm on which a wrist-worn device is worn, e.g., during the course of normal activity (e.g., stretching, putting items on a high shelf, etc.).

At 304, process 300 can identify a baseline time period during which the external pressure perturbation was not applied. In some implementations, process 300 may identify the baseline time period using one or more motion sensor(s) (which may include, e.g., one or more accelerometers and/or one or more gyroscopes), as shown in and described above in connection with FIG. 2. For example, process 300 may identify the baseline time period as one in which no motion of the wearable device was detected.

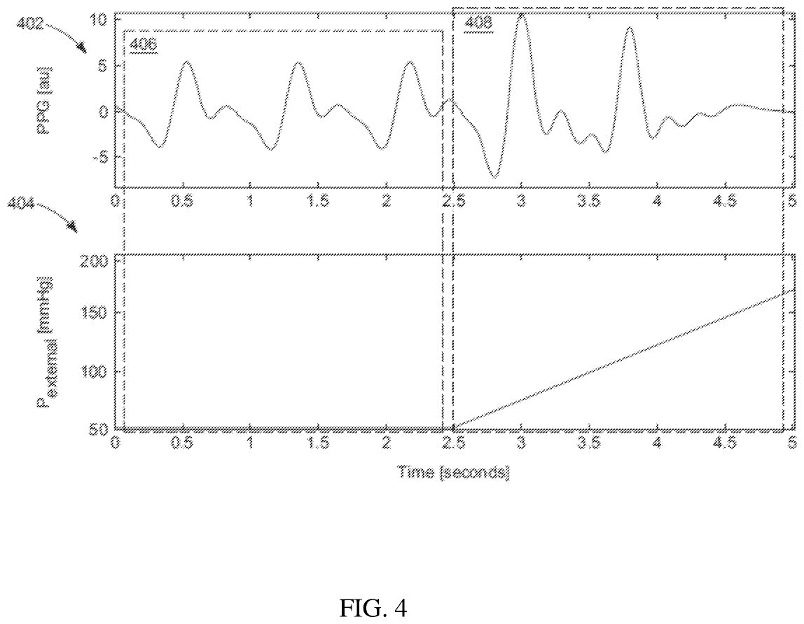

Turning to FIG. 4, an example of PPG data and pressure data collected during a period without external pressure perturbation (e.g., a baseline time period) and during a time period during which external pressure perturbation is applied are shown. Graph 402 shows measured PPG data over a time period of five seconds. Graph 404 shows pressure data measured concurrently with the PPG data. During baseline time period 406, no external pressure perturbation is applied. During time period 408, an external pressure perturbation, corresponding to a ramping increase in external pressure, is applied. Note that the PPG data during time period 408 differs from the PPG data during baseline time period 406, both in amplitude and shape.

Referring back to FIG. 3, at 306, process 300 can generate a synthetic plethysmograph waveform corresponding to a hypothetical plethysmograph waveform without external pressure perturbation based on the plethysmograph data corresponding to the baseline time period. In some examples, the synthetic plethysmograph waveform may be generated by replicating a subset of the measured plethysmograph data corresponding to the baseline time period. For example, in some embodiments, a predetermined number of plethysmograph cycles (e.g., one cycle, two cycles, three cycles, etc.) may be replicated to generate the synthetic plethysmograph waveform. The synthetic plethysmograph waveform may be generated to span substantially the same time period as the time period over which the measured plethysmograph data was obtained (e.g., with and without external pressure perturbation).

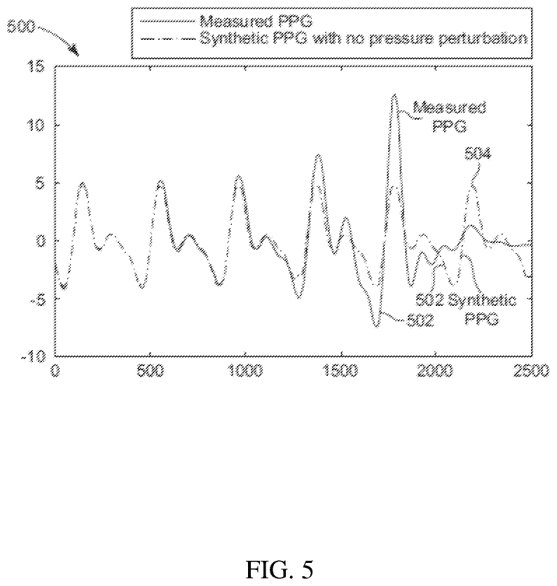

Turning to FIG. 5, an example of a synthetic plethysmograph waveform is shown in accordance with some embodiments. Graph 500 illustrates the measured PPG waveform 502 shown in and described above in connection with FIG. 4. Graph 500 also illustrates an example synthetic PPG waveform 504. Synthetic PPG waveform 504 is generated by replicating a subset of measured PPG waveform 502 from the baseline period during which no external pressure perturbation was applied.

Referring back to FIG. 3, at 308, process 300 can generate an arterial pressure waveform based on the synthetic plethysmograph waveform using one or more initial blood pressure values. In some embodiments, the one or more initial blood pressure values may include an initial systolic blood pressure value and/or an initial diastolic blood pressure value. In some embodiments, the one or more initial blood pressure values may include an initial mean blood pressure value. The arterial pressure waveform may be generated by scaling the synthetic plethysmograph waveform using the one or more initial blood pressure values. For example, the scaling may be a linear scaling. As a more particular example, in some embodiments, the synthetic plethysmograph waveform may be scaled such that a minimum of a particular cycle of the synthetic plethysmograph waveform corresponds to an initial diastolic blood pressure value, and such that a maximum of a particular cycle of the synthetic plethysmograph waveform corresponds to an initial systolic blood pressure value.

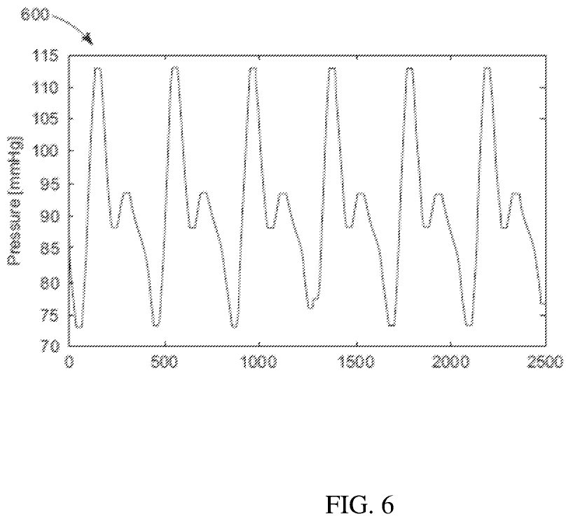

Turning to FIG. 6, an example of an arterial pressure waveform generated using the synthetic PPG waveform shown in and described above in connection with FIG. 5 is shown in accordance with some embodiments. As illustrated in graph 600, the arterial pressure waveform corresponds to a linear scaling of the synthetic PPG waveform of FIG. 5. In particular, the synthetic PPG waveform of FIG. 5 is scaled by an initial systolic blood pressure value and by an initial diastolic blood pressure value. Initial systolic blood pressure values and initial diastolic blood pressure values may be obtained or determined in any suitable manner, such as based on a pre-set value, historical measurements for the person, average measurements for a group of people, or the like.

Referring back to FIG. 3, at 310, process 300 can estimate a transmural pressure waveform based on the generated arterial pressure waveform and the pressure data (e.g., obtained at block 302). The transmural pressure waveform may indicate, at each time point, a difference between the arterial pressure (representing outwards pressure on the blood vessel walls) and the external applied pressure, as indicated in the pressure data. The transmural pressure waveform may be generated by determining a difference between the arterial pressure waveform generated at block 308 and the external pressure data obtained at block 302.

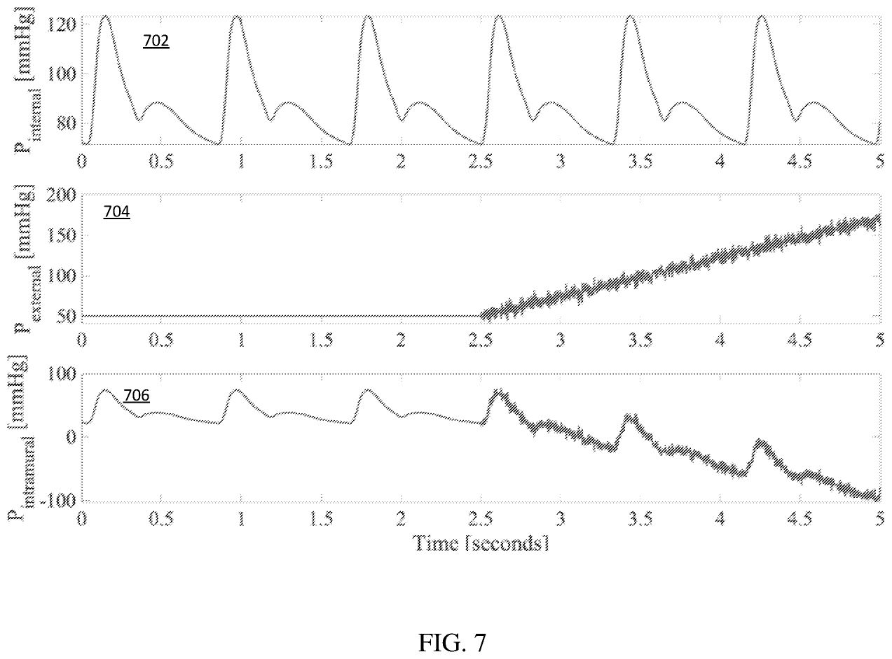

Turning to FIG. 7, an example transmural pressure waveform generated using the arterial pressure waveform shown in and described above in connection with FIG. 6 and the pressure data shown in and described above in connection with FIG. 4 is shown in accordance with some embodiments. Graph 702 illustrates the arterial pressure waveform shown in and described above in connection with FIG. 6. Graph 704 illustrates the external pressure data obtained at block 302 and shown in and described above in connection with FIG. 4. Graph 706 illustrates the determined transmural pressure waveform. In particular, graph 706 may be obtained by subtracting graph 704 (representing external pressure data) from graph 702 (the arterial pressure waveform).

Referring back to FIG. 3, at 312, process 300 can generate a predicted plethysmograph waveform using the transmural pressure waveform. The predicted plethysmograph waveform may correspond to the time period including and not including the external pressure perturbation. In other words, the predicted plethysmograph waveform may be considered a prediction of the plethysmograph data measured at block 302 that includes plethysmograph data without application of the external pressure perturbation and during application of the external pressure perturbation.

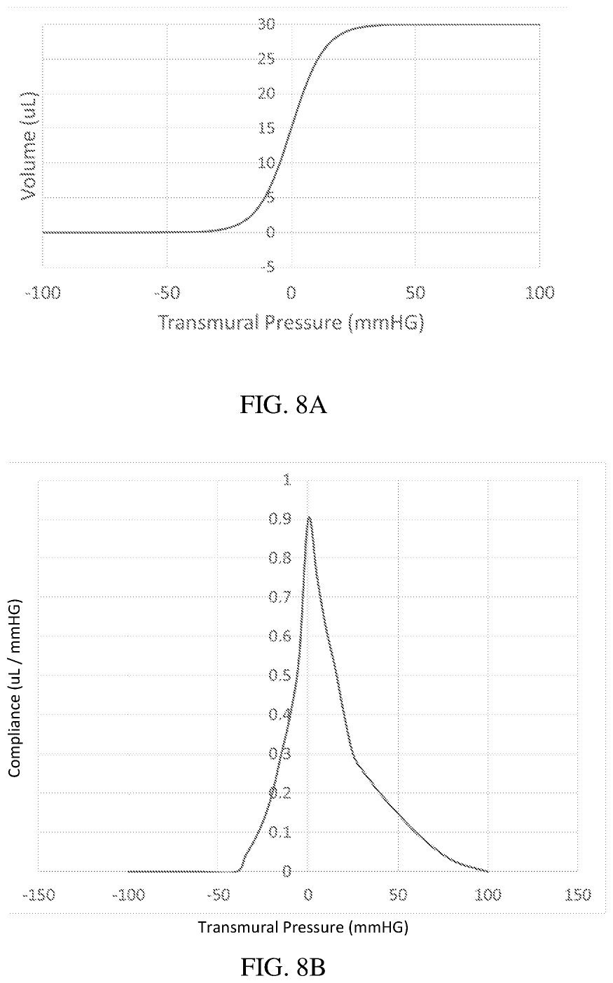

In some implementations, process 300 may generate the predicted plethysmograph waveform by providing data points of the transmural pressure waveform to a blood vessel compliance model that generates, as an output, a blood volume for a given transmural pressure. Aspects of an example of such a blood vessel compliance model are illustrated in FIGS. 8A and 8B. FIG. 8A depicts a graph that shows blood vessel volume (in microliters) as a function of transmural pressure (in mmHg). In some implementations (referring back to FIG. 3), process 300 may identify a blood volume by determining a blood volume corresponding to a given transmural pressure using a relationship similar to that depicted in FIG. 8A. FIG. 8B illustrates a relationship between blood vessel compliance (in microliters/mmHg) as a function of transmural pressure. In effect, FIG. 8B illustrates that blood vessel compliance is low at both extreme negative and positive transmural pressures, which is an intrinsic property of blood vessels.

Referring back to FIG. 3, in some implementations, process 300 may generate the predicted plethysmograph waveform by first determining, for a given transmural pressure data point of the transmural pressure waveform, a corresponding blood volume of the artery under consideration. Continuing with this example, in some implementations, because a plethysmograph data point represents blood volume in the blood vessel under consideration, process 300 may then generate plethysmograph data point of the predicted plethysmograph waveform using the blood volume determined using the transmural pressure data point. Process 300 may then repeat this process for the various transmural pressure data points of the transmural pressure waveform. An example of a blood vessel compliance model that may be utilized is described in Teng. Xiao-Fei and Zhang. Yuan-Ting “Theoretical Study on the Effect of Sensor Contact Force on Pulse Transit Time” IEEE Transactions on Biomedical Engineering, Vol. 54, No. 8, August 2007, which is hereby incorporated by reference herein in its entirety. In other words, blood volume of the arterial vessel may be used as a surrogate of non-zero frequency components of the PPG signal.

At 314, process 300 can determine whether to update the initial blood pressure values, e.g., those used to generate the predicted plethysmograph waveform. In some implementations, process 300 can determine whether to update the initial blood pressure values based on a difference between the predicted plethysmograph waveform and the measured plethysmograph data (e.g., as obtained at block 302).

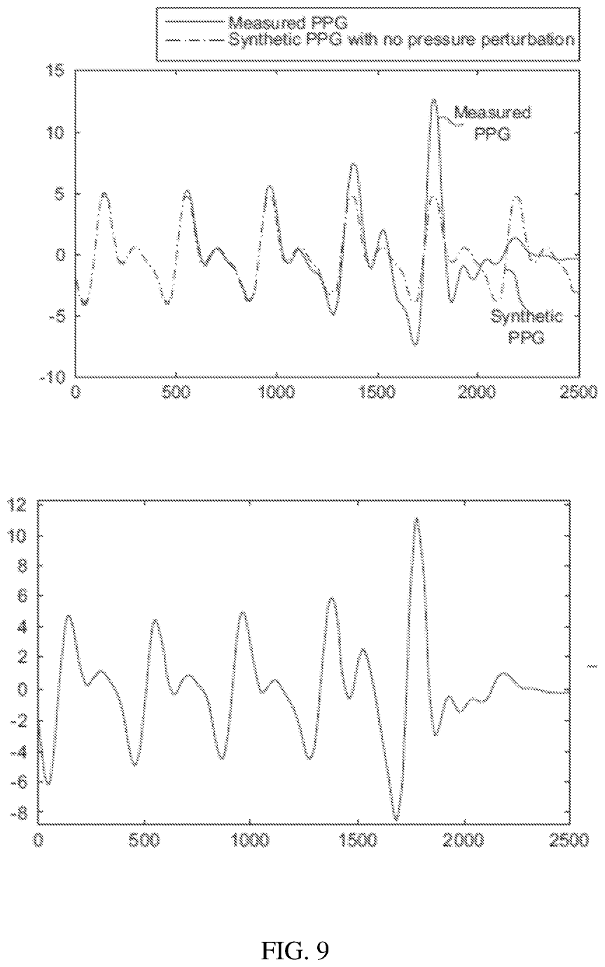

By way of illustration, FIG. 9 illustrates an example of measured PPG data and a predicted PPG waveform, e.g., predicted using the initial blood pressure values. Graph 902 depicts a measured PPG waveform (e.g., that shown in and described above in connection with FIG. 4).

Graph 904 depicts a corresponding predicted PPG waveform, which may be generated using initial blood pressure values and/or a transmural pressure waveform generated based on the initial blood pressure values, as described above. In some implementations, a determination of whether to update the initial blood pressure values may be made based on a difference between the measured PPG waveform depicted in graph 902 and the predicted PPG waveform depicted in graph 904.

Referring back to FIG. 3, process 300 may determine a difference between the predicted plethysmograph waveform and the measured plethysmograph data by determining an amplitude agnostic similarity score (e.g., a Pearson correlation coefficient, or the like) between the predicted and measured plethysmographs. In some implementations, process 300 may determine the initial blood pressure values are not to be updated responsive to determining that a difference between the predicted and measured plethysmograph waveforms are less than a predetermined threshold. Conversely, in some implementations, process 300 may determine the initial blood pressure values are to be updated responsive to determining that a difference between the predicted and measured plethysmograph waveforms exceed a predetermined threshold.

If, at 314, process 300 determines that the initial blood pressure values are not to be updated (“no” at 314), process 300 can proceed to 316 and can end.

Conversely, if, at 314, process 300 determines that the initial blood pressure values are to be updated (“yes” at 314), process 300 can proceed to 318 and can generate updated blood pressure values by optimizing a difference between the measured and predicted plethysmograph waveforms. For example, in some implementations, process 300 can use an optimization technique to determine updated blood pressure values that will generate a smaller difference between the measured and predicted plethysmograph waveforms. Examples of optimization techniques include gradient descent, expectation maximization, or the like.

Process 300 can then loop back to 308 and can generate a new arterial pressure waveform based on the synthetic plethysmograph waveform using the updated blood pressure values. In some embodiments, process 300 can loop through blocks 308-318 until process 300 determines that further updates to the blood pressure values are not needed. In some implementations, iterations through blocks 308-318 may be terminated responsive to a determination that more than a threshold number of iterations have already been completed, regardless of a difference between the measured and predicted plethysmograph waveforms.

It should be noted that, in some implementations, blood pressure values determined using the techniques described above in connection with FIG. 3 may be utilized to calibrate other blood pressure determination techniques configured to execute on the wearable device. By way of example, in some cases, a particular blood pressure technique, e.g., one that utilizes Pulse Transit Time (PTT), Pulse Wave Analysis (PWA) or the like, may determine relative blood pressure rather than absolute blood pressure. These techniques may be less computationally intensive. Accordingly, in some embodiments, the techniques described above may be used to determine absolute blood pressure values for a given user, which may be used to calibrate the relative blood pressures determined using other techniques. In some implementations, the techniques described herein may be utilized to initially determine absolute blood pressure values used to calibrate a second (e.g., a less computationally intensive) technique, at which time the second technique may be utilized by the wearable device.

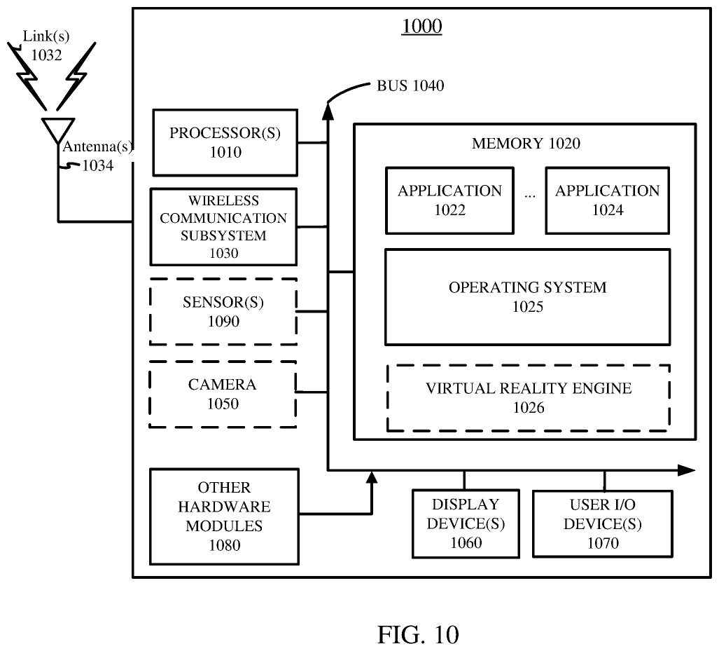

FIG. 10 is a simplified block diagram of an example of a computing system 1000 for

implementing some of the examples described herein. For example, in some embodiments, computing system may be used to implement a user device (e.g., a mobile phone, a tablet computer, a wrist-worn device, etc.) that implements the blocks of process 300 shown in and described above in connection with FIG. 3. In the illustrated example, computing system 1000 may include one or more processor(s) 1010 and a memory 1020. Processor(s) 1010 may be configured to execute instructions for performing operations at a number of components, and can be, for example, a general-purpose processor or microprocessor suitable for implementation within a portable electronic device. Processor(s) 1010 may be communicatively coupled with a plurality of components within computing system 1000. To realize this communicative coupling, processor(s) 1010 may communicate with the other illustrated components across a bus 1040. Bus 1040 may be any subsystem adapted to transfer data within computing system 1000. Bus 1040 may include a plurality of computer buses and additional circuitry to transfer data.

Memory 1020 may be coupled to processor(s) 1010. In some embodiments, memory 1020 may offer both short-term and long-term storage and may be divided into several units. Memory 1020 may be volatile, such as static random access memory (SRAM) and/or dynamic random access memory (DRAM) and/or non-volatile, such as read-only memory (ROM), flash memory, and the like. Furthermore, memory 1020 may include removable storage devices, such as secure digital (SD) cards. Memory 1020 may provide storage of computer-readable instructions, data structures, program modules, and other data for computing system 1000. In some embodiments, memory 1020 may be distributed into different hardware modules. A set of instructions and/or code might be stored on memory 1020. The instructions might take the form of executable code that may be executable by computing system 1000, and/or might take the form of source and/or installable code, which, upon compilation and/or installation on computing system 1000 (e.g., using any of a variety of generally available compilers, installation programs, compression/decompression utilities, etc.), may take the form of executable code.

In some embodiments, memory 1020 may store a plurality of application modules 1022 through 1024, which may include any number of applications. Examples of applications may include gaming applications, conferencing applications, video playback applications, or other suitable applications. The applications may include a depth sensing function or eye tracking function. Application modules 1022-1024 may include particular instructions to be executed by processor(s) 1010. In some embodiments, certain applications or parts of application modules 1022-1024 may be executable by other hardware modules 1080. In certain embodiments, memory 1020 may additionally include secure memory, which may include additional security controls to prevent copying or other unauthorized access to secure information.

In some embodiments, memory 1020 may include an operating system 1025 loaded therein. Operating system 1025 may be operable to initiate the execution of the instructions provided by application modules 1022-1024 and/or manage other hardware modules 1080 as well as interfaces with a wireless communication subsystem 1030 which may include one or more wireless transceivers. Operating system 1025 may be adapted to perform other operations across the components of computing system 1000 including threading, resource management, data storage control and other similar functionality.

Wireless communication subsystem 1030 may include, for example, an infrared communication device, a wireless communication device and/or chipset (such as a Bluetooth® device, an IEEE 802.11 device, a Wi-Fi device, a WiMax device, cellular communication facilities, etc.), and/or similar communication interfaces. Computing system 1000 may include one or more antennas 1034 for wireless communication as part of wireless communication subsystem 1030 or as a separate component coupled to any portion of the system. Depending on desired functionality, wireless communication subsystem 1030 may include separate transceivers to communicate with base transceiver stations and other wireless devices and access points, which may include communicating with different data networks and/or network types, such as wireless wide-arca networks (WWANs), wireless local area networks (WLANs), or wireless personal area networks (WPANs). A WWAN may be, for example, a WiMax (IEEE 802.17) network. A WLAN may be, for example, an IEEE 802.11x network. A WPAN may be, for example, a Bluetooth network, an IEEE 802.7x, or some other types of network. The techniques described herein may also be used for any combination of WWAN, WLAN, and/or WPAN. Wireless communications subsystem 1030 may permit data to be exchanged with a network, other computer systems, and/or any other devices described herein. Wireless communication subsystem 1030 may include a means for transmitting or receiving data, such as identifiers of HMD devices, position data, a geographic map, a heat map, photos, or videos, using antenna(s) 1034 and wireless link(s) 1032. Wireless communication subsystem 1030, processor(s) 1010, and memory 1020 may together comprise at least a part of one or more of a means for performing some functions disclosed herein.

Embodiments of computing system 1000 may also include one or more sensors 1090. Sensor(s) 1090 may include, for example, an image sensor, an accelerometer, a force sensor, a hydrostatic pressure sensor, a temperature sensor, a proximity sensor, a magnetometer, a gyroscope, an inertial sensor (e.g., a module that combines an accelerometer and a gyroscope), an ambient light sensor, or any other similar module operable to provide sensory output and/or receive sensory input, such as a depth sensor or a position sensor. For example, in some implementations, sensor(s) 1090 may include one or more inertial measurement units (IMUs) and/or one or more position sensors. An IMU may generate calibration data indicating an estimated position of a device, based on measurement signals received from one or more of the position sensors. A position sensor may generate one or more measurement signals in response to motion of a device. Examples of the position sensors may include, but are not limited to, one or more accelerometers, one or more gyroscopes, one or more magnetometers, another suitable type of sensor that detects motion, a type of sensor used for error correction of the IMU, or some combination thereof. The position sensors may be located external to the IMU, internal to the IMU, or some combination thereof. At least some sensors may use a structured light pattern for sensing.

Computing system 1000 may include a display module 1060. Display module 1060 may be a near-eye display, and may graphically present information, such as images, videos, and various instructions, from computing system 1000 to a user. Such information may be derived from one or more application modules 1022-1024, virtual reality engine 1026, one or more other hardware modules 1080, a combination thereof, or any other suitable means for resolving graphical content for the user (e.g., by operating system 1025). Display module 1060 may use liquid crystal display (LCD) technology, light-emitting diode (LED) technology (including, for example, OLED, ILED, μLED, AMOLED, TOLED, etc.), light emitting polymer display (LPD) technology, or some other display technology.

Computing system 1000 may include a user input/output module 1070. User input/output module 1070 may allow a user to send action requests to computing system 1000. An action request may be a request to perform a particular action. For example, an action request may be to start or end an application or to perform a particular action within the application. User input/output module 1070 may include one or more input devices. Example input devices may include a touchscreen, a touch pad, microphone(s), button(s), dial(s), switch(es), a keyboard, a mouse, a game controller, or any other suitable device for receiving action requests and communicating the received action requests to computing system 1000. In some embodiments, user input/output module 1070 may provide haptic feedback to the user in accordance with instructions received from computing system 1000. For example, the haptic feedback may be provided when an action request is received or has been performed.

Computing system 1000 may include a camera 1050 that may be used to take photos or videos. Camera 1050 may be configured to take photos or videos of the user. Camera 1050 may also be used to take photos or videos of the environment, for example, for VR. AR, or MR applications. Camera 1050 may include, for example, a complementary metal-oxide-semiconductor (CMOS) image sensor with a few millions or tens of millions of pixels. In some implementations, camera 1050 may include two or more cameras that may be used to capture 3-D images.

In some embodiments, computing system 1000 may include a plurality of other hardware modules 1080. Each of other hardware modules 1080 may be a physical module within computing system 1000. While each of other hardware modules 1080 may be permanently configured as a structure, some of other hardware modules 1080 may be temporarily configured to perform specific functions or temporarily activated. Examples of other hardware modules 1080 may include, for example, an audio output and/or input module (e.g., a microphone or speaker), a near field communication (NFC) module, a rechargeable battery, a battery management system, a wired/wireless battery charging system, etc. In some embodiments, one or more functions of other hardware modules 1080 may be implemented in software.

In some embodiments, memory 1020 of computing system 1000 may also store a virtual reality engine 1026. Virtual reality engine 1026 may execute applications within computing system 1000 and receive position information, acceleration information, velocity information, predicted future positions, or some combination thereof from the various sensors. In some embodiments, the information received by virtual reality engine 1026 may be used for producing a signal (e.g., display instructions) to display module 1060. For example, if the received information indicates that the user has looked to the left, virtual reality engine 1026 may generate content that mirrors the user's movement in a virtual environment. Additionally, virtual reality engine 1026 may perform an action within an application in response to an action request received from user input/output module 1070 and provide feedback to the user. The provided feedback may be visual, audible, or haptic feedback. In some implementations, processor(s) 1010 may include one or more GPUs that may execute virtual reality engine 1026.

In various implementations, the above-described hardware and modules may be implemented on a single device or on multiple devices that can communicate with one another using wired or wireless connections. For example, in some implementations, some components or modules, such as GPUs, virtual reality engine 1026, and applications (e.g., tracking application), may be implemented on two or more paired or connected devices.

In alternative configurations, different and/or additional components may be included in computing system 1000. Similarly, functionality of one or more of the components can be distributed among the components in a manner different from the manner described above. For example, in some embodiments, computing system 1000 may be modified to include other system environments, such as an AR system environment and/or an MR environment.

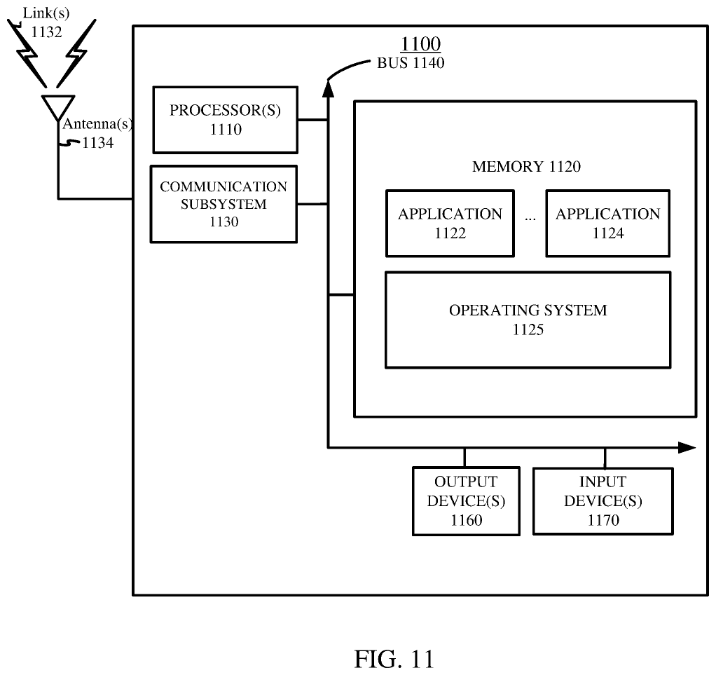

FIG. 11 is a simplified block diagram of an example of a computing system 1100 that may be implemented in connection with a server in accordance with some embodiments. For example, computing system 1100 may be used to implement a server used to generate a model or portions of a model that predicts blood pressure based on measured plethysmography data, or the like.

In the illustrated example, computing system 1100 may include one or more processor(s) 1110 and a memory 1120. Processor(s) 1110 may be configured to execute instructions for performing operations at a number of components, and can be, for example, a general-purpose processor or microprocessor suitable for implementation within a portable electronic device. Processor(s) 1110 may be communicatively coupled with a plurality of components within computing system 1100. To realize this communicative coupling, processor(s) 1110 may communicate with the other illustrated components across a bus 1140. Bus 1140 may be any subsystem adapted to transfer data within computing system 1100. Bus 1140 may include a plurality of computer buses and additional circuitry to transfer data. In some embodiments, processor(s) 1110 may be configured to perform one or more blocks of process 300, as shown in and described above in connection with FIG. 3, respectively.

Memory 1120 may be coupled to processor(s) 1110. In some embodiments, memory 1120 may offer both short-term and long-term storage and may be divided into several units. Memory 1120 may be volatile, such as static random access memory (SRAM) and/or dynamic random access memory (DRAM) and/or non-volatile, such as read-only memory (ROM), flash memory, and the like. Furthermore, memory 1120 may include removable storage devices, such as secure digital (SD) cards. Memory 1120 may provide storage of computer-readable instructions, data structures, program modules, and other data for computing system 1100. In some embodiments, memory 1120 may be distributed into different hardware modules. A set of instructions and/or code might be stored on memory 1120. The instructions might take the form of executable code that may be executable by computing system 1100, and/or might take the form of source and/or installable code, which, upon compilation and/or installation on computing system 1100 (e.g., using any of a variety of generally available compilers, installation programs, compression/decompression utilities, etc.), may take the form of executable code.

In some embodiments, memory 1120 may store a plurality of application modules 1122 through 1124, which may include any number of applications. Examples of applications may include gaming applications, conferencing applications, video playback applications, or other suitable applications. Application modules 1122-1124 may include particular instructions to be executed by processor(s) 1110. In some embodiments, certain applications or parts of application modules 1122-1124 may be executable by other hardware modules. In certain embodiments, memory 1120 may additionally include secure memory, which may include additional security controls to prevent copying or other unauthorized access to secure information.

In some embodiments, memory 1120 may include an operating system 1125 loaded therein. Operating system 1125 may be operable to initiate the execution of the instructions provided by application modules 1122-1124 and/or manage other hardware modules as well as interfaces with a wireless communication subsystem 1130 which may include one or more wireless transceivers. Operating system 1125 may be adapted to perform other operations across the components of computing system 1100 including threading, resource management, data storage control and other similar functionality.

Communication subsystem 1130 may include, for example, an infrared communication device, a wireless communication device and/or chipset (such as a Bluetooth® device, an IEEE 802.11 device, a Wi-Fi device, a WiMax device, cellular communication facilities, etc.), a wired communication interface, and/or similar communication interfaces. Computing system 1100 may include one or more antennas 1134 for wireless communication as part of wireless communication subsystem 1130 or as a separate component coupled to any portion of the system. Depending on desired functionality, communication subsystem 1130 may include separate transceivers to communicate with base transceiver stations and other wireless devices and access points, which may include communicating with different data networks and/or network types, such as wireless wide-area networks (WWANs), wireless local area networks (WLANs), or wireless personal area networks (WPANs). A WWAN may be, for example, a WiMax (IEEE 802.17) network. A WLAN may be, for example, an IEEE 802.11x network. A WPAN may be, for example, a Bluetooth network, an IEEE 802.8x, or some other types of network. The techniques described herein may also be used for any combination of WWAN, WLAN, and/or WPAN. Communications subsystem 1130 may permit data to be exchanged with a network, other computer systems, and/or any other devices described herein. Communication subsystem 1130 may include a means for transmitting or receiving data, using antenna(s) 1134, wireless link(s) 1132, or a wired link. Communication subsystem 1130, processor(s) 1110, and memory 1120 may together comprise at least a part of one or more of a means for performing some functions disclosed herein.

In some embodiments, computing system 1100 may include one or more output device(s) 1160 and/or one or more input device(s) 1170. Output device(s) 1170 and/or input device(s) 1170 may be used to provide output information and/or receive input information.

Embodiments disclosed herein may be used to implement components of an artificial reality system or may be implemented in conjunction with an artificial reality system. Artificial reality is a form of reality that has been adjusted in some manner before presentation to a user, which may include, for example, a virtual reality, an augmented reality, a mixed reality, a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, for example, create content in an artificial reality and/or are otherwise used in (e.g., perform activities in) an artificial reality. The artificial reality system that provides the artificial reality content may be implemented on various platforms, including an HMD connected to a host computer system, a standalone HMD, a mobile device or computing system, or any other hardware platform capable of providing artificial reality content to one or more viewers.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, systems, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing various embodiments. Various changes may be made in the function and arrangement of elements without departing from the spirit and scope of the present disclosure.

Also, some embodiments were described as processes depicted as flow diagrams or block diagrams. Although each may describe the operations as a sequential process, many of the operations may be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It will be apparent to those skilled in the art that substantial variations may be made in accordance with specific requirements. For example, customized or special-purpose hardware might also be used, and/or particular elements might be implemented in hardware, software (including portable software, such as applets, etc.), or both. Further, connection to other computing devices such as network input/output devices may be employed.

With reference to the appended figures, components that can include memory can include non-transitory machine-readable media. The term “machine-readable medium” and “computer-readable medium” may refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media such as compact disk (CD) or digital versatile disk (DVD), punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code. A computer program product may include code and/or machine-executable instructions that may represent a procedure, a function, a subprogram, a program, a routine, an application (App), a subroutine, a module, a software package, a class, or any combination of instructions, data structures, or program statements.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that are also expected to depend at least in part upon the context in which such terms are used. Typically, “or” if used to associate a list, such as A, B, or C, is intended to mean A, B, and C. here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics.

However, it should be noted that this is merely an illustrative example and claimed subject matter is not limited to this example. Furthermore, the term “at least one of” if used to associate a list, such as A, B, or C. can be interpreted to mean any combination of A, B, and/or C, such as A, AB, AC, BC. AA, ABC, AAB, AABBCCC, etc.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific embodiments have been described, these are not intended to be limiting. Various modifications and equivalents are within the scope of the following claims.

What is claimed is:

1. A method of determining blood pressure, the method comprising:
obtaining measured plethysmograph data and pressure data that corresponds to a time period comprising a first portion during which an external pressure perturbation was not applied and a second portion during which the external pressure perturbation was applied;
generating predicted plethysmograph data using: (i) initial blood pressure values; (ii) at least a subset of the measured plethysmograph data associated with the first portion during which the external pressure perturbation was not applied; and (iii) the pressure data; and
modifying the initial blood pressure values based on a difference between the predicted plethysmograph data and the measured plethysmograph data to generate final blood pressure values.

2. The method of claim 1, wherein generating the predicted plethysmograph comprises:
generating a synthetic arterial pressure waveform based on at least a subset of the measured plethysmograph data associated with the first portion during which the external pressure perturbation was not applied and using initial blood pressure values, wherein the synthetic arterial pressure waveform represents an internal blood vessel pressure without any external pressure perturbation; and
modifying the synthetic arterial pressure waveform to generate a transmural pressure waveform using at least the pressure data, wherein the predicted plethysmograph data is generated using the transmural pressure waveform.

3. The method of claim 2, wherein generating the synthetic arterial pressure waveform comprises:
generating synthetic plethysmograph data based on the subset of the plethysmograph data associated with the first portion during which the external pressure perturbation was not applied; and
scaling the synthetic plethysmograph data using the initial blood pressure values.

4. The method of claim 2, wherein generating the predicted plethysmograph data comprises applying the transmural pressure waveform to a blood vessel elasticity model that generates, for a given transmural pressure, a corresponding blood volume of the blood vessel.

5. The method of claim 1, wherein the predicted plethysmograph data indicates changes to a shape of a cycle within the plethysmograph data due to the external pressure perturbation.

6. The method of claim 1, wherein the first portion during which the external pressure perturbation is not applied is identified using data from at least one motion sensor.

7. The method of claim 1, wherein modifying the initial blood pressure values based on the difference between the predicted plethysmograph data and the measured plethysmograph data comprises using an iterative optimization algorithm to obtain the final blood pressure values.

8. The method of claim 1, wherein the external pressure perturbation comprises at least one of: an externally applied compression force; or a change in hydrostatic pressure.

9. The method of claim 1, wherein the measured plethysmograph data is obtained using at least one light source and light detector disposed in or on a wearable device, and wherein the pressure data is obtained from at least one force sensor disposed in or on the wearable device.

10. The method of claim 1, further comprising, prior to obtaining the measured plethysmograph data and the pressure data, presenting an instruction to a user to apply the external pressure perturbation.

11. The method of claim 1, wherein the time period is less than about 5 seconds.

12-20. (canceled)
